(19) United States
(12) Patent Application Publication
(10) Pub. No.: US 2006/0224131 A1
(43) Pub. Date: Oct. 5, 2006

(54) STOMA DEVICES AND DRESSINGS AND METHODS THEREFOR

(76) Inventor: Deborah Calvert, Chorley (GB)

Correspondence Address: ADAMS EVANS P.A., 301 SOUTH TRYON STREET, SUITE 2180, TWO WACHOVIA CENTER, CHARLOTTE, NC, 28282-1991, US

(21) Appl. No.: 11/277,571

(22) Filed: Mar. 27, 2006

(30) Foreign Application Priority Data
Mar. 30, 2005 (GB) 0506343.3

(51) Int. Cl. A61F 5/44

(52) U.S. Cl. 604/344

(57) ABSTRACT

A stoma device for maintaining a gastrostomy and/or urostomy stoma includes a deformable stem having opposed first and second ends which is arranged to be inserted through a stoma. The device has a head which is connected to the stem at its first end and arranged to restrict migration of the stoma device through the stoma. Also disclosed are a method of maintaining a gastric or urostomy stoma and a medical dressing.

### FIELD OF THE INVENTION

The present invention relates to stoma devices, particularly, though not exclusively, to stoma devices for maintaining gastrostomy stomas, and to methods of maintaining stomas with such devices. The present invention also relates to adhesive dressings.

### BACKGROUND TO THE INVENTION

There are known a number of methods of artificially supplying nourishment to patients. One such method comprises forming an incision through the abdominal and stomach walls to create a stoma (a gastrostomy). To provide nourishment a hollow tube (gastrostomy tube) is passed through the stoma such that the tube has an opening within the stomach. Liquid feed may then be supplied through the tube into the stomach. It is important that the gastrostomy tube remains in place to maintain the stoma and to prevent gastric acid egressing through the stoma. However, gastromy tubes may become accidentally removed. For example, some known gastrostomy tubes use a balloon located within the stomach to hold them in place, if this becomes deflated the tube may fall out. Damage of the balloon which leads to deflation thereof may for example be caused by Candidas infection.

If a tube should become removed it is important that it is replaced speedily to stop gastric acid from exiting the stoma and chemically burning the surrounding skin. Additionally, such stomas are quick to heal and can seal up within a matter of hours. Thus, if a gastromy tube is not quickly reinserted a patient may need to undergo a further gastrostomy operation.

The insertion of a gastrostomy tube is not a simple process and must be performed by a qualified medical practitioner. A gastrostomy tube may though fall out when such a medical practitioner is not available. It is thus known to employ temporary means to maintain the stoma until a gastrostomy tube can be reinserted. One known method employs a Foley Catheter. However such catheters are not intended for this use and there is a risk that they could migrate into and beyond the stomach or become removed themselves.

Accordingly, the present invention aims to address at least one disadvantage associated with the prior art whether discussed herein or otherwise.

### SUMMARY OF THE INVENTION

According to a first aspect of the present invention there is provided a stoma device for maintaining a gastrostomy and/or urostomy stoma wherein said device comprises a deformable stem having opposed first and second ends, and which is arranged to be inserted through a stoma, and a head which is connected to the stem at its first end and arranged to restrict migration of the stoma device through the stoma.

Preferably, the device is arranged to maintain a gastrostomy stoma.

Suitably, the stem is resiliently deformable.

The stem of the device may be deformable such that, in use, should the free end (second end) of the stem abut an internal body wall, for example the stomach wall, the stem can deform so as to minimise any risk of causing internal injury.

Suitably, the second end of the stem is occluded. Suitably, the second end of the stem is rounded. Thus, any risk of stem causing internal injury to a patient may be minimised. Suitably, the stem has a rounded second end to minimise the risk of damaging or perforating the stoma mucosa.

Preferably, the stem is compressible. Preferably, the stem is compressible in a direction transverse to the axis of the stem.

Suitably, the stem comprises a hollow tube.

Preferably, the stem is bendable.

Suitably, the stem comprises means arranged to control the point at which the stem bends. Suitably, said means comprises one or more weak points along the stems length. A weak point may comprise a point along the stems length at which the stem is more prone to collapse.

A weak point may comprise a thinning of the stem. Suitably, a weak point comprises an aperture in a side wall of the stem.

Suitably, the stem is arranged to bend at a point internal of a patients stomach wall. Thus, should a force, such as that caused by internal or external pressure, tend to push the stem through the stoma the stem may instead bend. If bent, in use, the stem may hook around the stomach wall to restrict the stem from exiting the stoma.

Suitably, the stem comprises a side wall which defines a chamber. Suitably the chamber is an open chamber. Suitably, one or more openings to the chamber are provided in a side wall or at a first end of the stem but not in both. Thus, although the chamber may be open it may not provide a path for fluid to travel through the stoma device either into or out of a patients body.

Suitably, the device is arranged such that, in use, there may be an opening into the device lying internal or external of a patient but not both and the device may thus not provide a pathway through the stoma.

Thus, in use, the device may occlude a stoma. This may be important for a number of reasons. For example, whilst it is intended that the device be inserted through both the abdominal and stomach walls a device may, on rare occasions, be incorrectly inserted such that the second end lies in the peritinium (between the stomach and abdominal walls). When so located the device may still provide a benefit by maintaining the abdominal stoma and leaving a surgeon only the stomach stoma to recreate. However, if feed were introduced into the peritinium it could be harmful to a patient. A device which occludes a stoma may though prevent feed being introduced to the peritinium and thus may guard against injury to the patient.

Suitably, if the first end of the stem is provided with an opening then a corresponding opening is provided through the head of the device.

Preferably, the first end of the stem is occluded. The first end of the stem may be occluded by the head of the device.

Preferably, the side wall of the stem comprises one or more openings. Suitably, the side wall of the stem comprises one or more apertures arrange to lie between 10 and 90% of the stems length from the first end of the stem, more preferably between 30 and 70% of the stems length from the first end.

Suitably, the side wall of the stem comprises two apertures. Suitably, said apertures lie on opposed sides of the stem at substantially the same point along the stems length.

Said apertures suitably provide weak points along the stems longitudinal extent which may control the point at which the stem bends.

Suitably, the head comprises one or more retaining members extending substantially transverse to the stems axis. Preferably, the head comprises a retaining member which comprises a disc connected to the stems first end.

Preferably the head comprises handle means by which the device can be held.

Suitably the handle means comprises a button. The button may be cylindrical. Suitably, the button extends along the axis of the stem. Suitably the retaining member or members extend further from the stems axis in a radial direction than does the button.

Suitably, the device comprises adhesive attachment means for attaching the device to a patient.

The adhesive attachment means may comprise an adhesive tape or dressing attached to the head. Suitably, an adhesive tape or dressing is attached to a retaining member.

Preferably, the device comprises an adhesive dressing arranged to fit around a button of the device and sit over one or more retaining members.

Suitably, the adhesive dressing extends further from the stems axis in a radial direction than does the retaining member or members.

Suitably, the adhesive dressing comprises one or more wings for adhering to a patients skin.

Suitably, the adhesive dressing comprises an annular disc for attaching to the head and from which extend one or more wings for adhering to a patients skin. Suitably, the adhesive dressing comprises two or more, preferably two wings.

The dressing may comprise an absorbent layer, which may be a gauze, carrying an adhesive. The dressing may comprise a removable cover sheet for covering the adhesive prior to the device being used.

Suitably, the dressing is arranged to stretch in a radial direction relative to the stem.

The dressing suitably comprises a plurality of elongate slits. Preferably, the dressing comprises generally annularly extending slits. Suitably, the dressing comprises slits arranged in concentric arcs. Suitably slits of one arc are aligned with solid sections of an adjacent arc and vice versa. Thus, when stretched radially the slits may open out and the dressing may adopt a lattice structure.

Thus, when the skin around a stoma is stretched, for example by inter abdominal movement or compressed by external forces the dressing may accommodate such movement. Thus, the stoma device may be retained in position more readily than if a known adhesive dressing were used. By expanding the dressing may reduce pressure at the stoma site and may thus minimise necrotic damage.

Suitably, the stem comprises a resiliently deformable material. Suitably, the stem comprises a plastics material, most preferably polyurethane. The head may comprise the same material as the stem. Suitably the head and stem are integral.

Suitably the stem has a length of between 3 cm and 8 cm, preferably of between 3.5 cm and 6.5 cm, for example around 6 cm.

Suitably, the stem has a diameter of between 0.2 cm and 2 cm, preferably of between 0.4 and 1 cm. Suitably, the stem comprises a diameter corresponding to a known French size. Thus, in use, should a gastrostomy tube of a given French size become removed a device of a corresponding French size may be used to maintain the Stoma. Thus, a gastrostomy tube having the same French size as the original tube may subsequently be reinserted through the stoma.

According to a second aspect of the present invention there is provided a method of maintaining a gastric or urostomy stoma wherein the method employs a stoma device according to the first aspect and wherein the method comprises inserting a stem of the device through the stoma.

Suitably, the method comprises inserting the stem until the head abuts the skin adjacent the stoma and then adhering the device to the skin.

Suitably, the method comprises a method of maintaining a gastric stoma. The method may be suitable for maintaining a stoma if a gastrostomy tube becomes removed, for example if the tube falls out, until such time as a medical practitioner can reinsert a gastrostomy tube.

Suitably, the method comprises inserting the stem through a stoma in the abdomen wall. Preferably, the method further comprises inserting the stem through a stoma in the stomach wall.

Suitably, the method can be performed by a person who does not have specific training.

Suitably, the method can be performed by a person who does not have medical training. For example the method may be performed by a patients relative.

The method may provide a temporary means for maintaining a stoma and may not constitute a therapeutic or surgical treatment.

Suitably, a medical practitioner can remove the stoma device subsequent to said method being performed in order to reinsert a gastrostomy tube.

According to a third aspect of the present invention there is provided a medical dressing having elongate slits formed therein such that, in use, the dressing can be stretched in a direction substantially transverse to the longitudinal extent of the slits.

In use, when the dressing is stretched transverse to the longitudinal extent of the slits the slits may open out to adopt a generally diamond shaped form. The dressing may thus have a lattice structure when stretched.

Suitably, the dressing comprises an absorbent layer, which may be a gauze, carrying an adhesive. The dressing may comprise a removable cover sheet for covering the adhesive prior to the dressing being used.

Suitably, the dressing comprises a plurality of elongate slits which extend generally parallel to one another.

Suitably, the dressing comprises a plurality of slits lying on a first line and a plurality of slits lying on one or more further lines which suitably extend generally parallel to the first line. Suitably, the dressing comprises three or more such lines of slits. Suitably slits of one line are aligned with solid sections of an adjacent line and vice versa.

The dressing may comprise arcuate lines of slits. Suitably, the dressing comprises slits arranged in concentric arcs. Suitably slits of one arc are aligned with solid sections of an adjacent arc and vice versa.

The dressing may comprise any feature as described in relation to the first aspect.

### BRIEF DESCRIPTION OF THE DRAWINGS

The present invention will now be described by way of example, with reference to the drawings that follow, in which:

FIG. 1 is a perspective view of a stoma device;

FIG. 2 is a cross section showing the stoma device of FIG. 1 in use;

FIG. 3 is a plan view showing the stoma device of FIG. 1; and

FIG. 4 is a plan view showing a dressing.

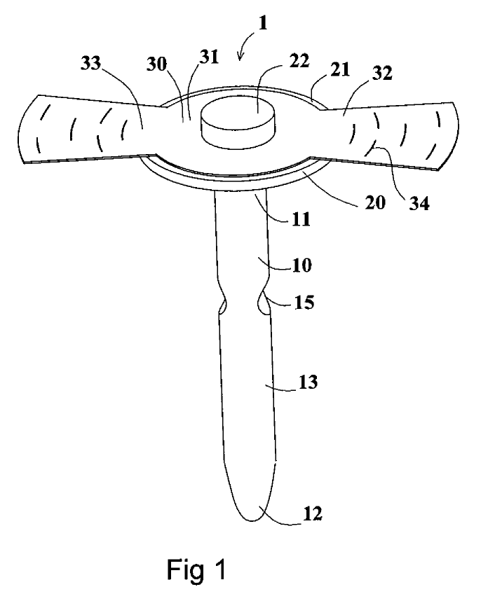

As illustrated by FIGS. 1 to 3 a stoma device 1 comprises a deformable stem 10 having opposed first and second ends 11, 12 and a head 20 formed at the first end 11 of the stem 10.

The device is arranged to maintain a stoma with the stem 10 being arranged to be passed through a stoma and the head 10 being arranged to prevent the device 1 migrating into the body.

The stem 10 comprises a hollow tube having a side wall 13 defining a cavity 14. The stem 10 is both compressible and bendable.

The second end 12 of the stem 10 is occluded and rounded so as to minimise risk of causing injury in use.

Apertures 15 are provided in the stem 10 to form openings into the cavity 14. These apertures 15 create weak points in the stem 10 and thus increase the likelihood of the stem 10 bending at that point. Thus, in use if an internal or external abdominal force causes the second end 12 of the stem to be pushed towards the stoma the stem may bend rather than be forced through the stoma. This bending may also prevent the end 12 damaging the stomach wall.

The first end 11 of the stem 10 is occluded by the head 20. Thus, there is no through path for fluid to pass through the stoma.

The head 20 comprises a retaining member in the form of an annular disc 21 and handle means in the form of a button 22 which are coaxial with the stem 10.

Secured to the retaining member is an adhesive dressing 30 for adhering to a patients skin 4 in use.

The adhesive dressing 30 comprises an annular disc 31 surrounding the button and two opposed wings 32, 33 extending beyond the retaining member disc 21 in a radial direction relative to the stem 10.

The dressing 30 comprises an absorbent gauze carrying an adhesive and having a plurality of generally annularly extending slits 34 formed therein. Thus, in use, the dressing may stretch in a direction generally transverse to the slits longitudinal extent to accommodate stretching of the skin such as caused by internal or external abdominal pressure. This may improve patient comfort and reduce movement of the device 1 relative to the stoma which may therefore reduce any risk to the patient.

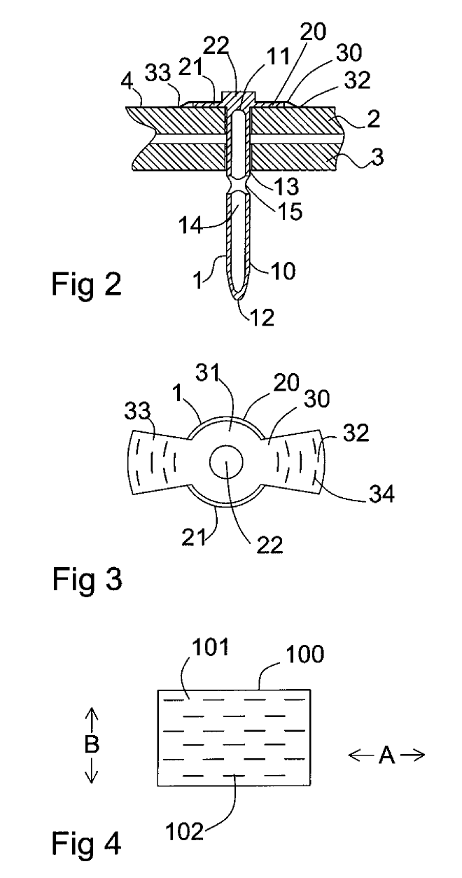

As illustrated by FIG. 2, if a gastrostomy tube becomes removed a device 1 can be used to maintain the stoma. The device 1 can be held by the button 22 and can be inserted into the stoma such that the stem 10 passes through the abdominal wall 2 and stomach wall 3. The device is inserted until the disc 21 of the head 20 abuts the skin 4 adjacent the stoma. In use, the head 20 then prevents the device 1 migrating into the stomach.

when the device 1 is in place the adhesive dressing 30 may be pressed onto the skin 4 to fix the device 1 in position. To maintain the dressings adhesive qualities prior to use the dressing 30 comprises a removable cover sheet (not shown) for covering the adhesive.

FIG. 4 illustrates a dressing 100 having a similar construction to that of the dressing 30 employed in the stoma device.

The dressing 100 comprises an absorbent layer 101, which comprises a gauze, and which carries an adhesive (not shown) on one side thereof. To maintain the dressings adhesive qualities prior to use the dressing comprises a removable cover sheet (not shown) for covering the adhesive.

The dressing 100 comprises a plurality of elongate slits 102 arranged to extend in generally parallel lines orientated in a first direction (arrows A). The slits of one line are aligned with solid parts of an adjacent line of slits and vice versa. In use, when applied to the skin, if the skin stretches generally transverse to the slit longitudinal extent (for example in the direction of arrows B) then the slits can open out and may adopt a generally diamond form. This may allow the dressing to stretch with the skin and may improve the dressings effectiveness and patient comfort.

It will be appreciated that preferred embodiments of the present invention may enhance stoma care.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiments). The invention extends to any novel one, or any novel combination, of the features disclosed in this specification (including any accompanying claims, abstract and drawings), or to any novel one, or any novel combination, of the steps of any method or process so disclosed.

What is claimed is:

1. A device for maintaining a stoma in a patient's body wherein said device comprises a deformable stem having opposed first and second ends, and which is arranged to be inserted through a stoma, and a head which is connected to the stem at its first end and arranged to restrict migration of the stoma device through the stoma.

2. A device according to claim 1, wherein the device is arranged to maintain a gastrostomy and/or urostomy stoma.

3. A device according to claim 1, wherein the second end of stem is occluded and rounded.

4. A device according to claim 1, wherein the stem comprises a hollow tube.

5. A device according to claim 1, wherein the stem comprises a side wall which defines a chamber.

6. A device according to claim 5, wherein the chamber is an open chamber and at least one opening to the chamber is provided in a side wall or at a first end of the stem but not in both.

7. A device according to claim 6, wherein the first end of the stem is occluded and the side wall of the stem comprises at least one opening.

8. A device according to claim 1, where the stem is bendable.

9. A device according to claim 8, wherein the stem comprises means arranged to control the point at which the stem bends.

10. A device according to claim 9, wherein said means comprises at least one weak points along the stem's length.

11. A device according to claim 10, wherein a weak point comprises an aperture in a side wall of the stem.

12. A device according to claim 1, wherein the stem is compressible.

13. A device according to claim 1, wherein the stem has a length of between 3 cm and 8 cm and a diameter of between 0.2 cm and 2 cm.

14. A device according to claim 1, wherein the head comprises at least one retaining member extending substantially transverse to the stem's axis.

15. A device according to claim 1, wherein the head comprises handle means by which the device can be held.

16. A device according to claim 1 wherein the device comprises adhesive attachment means for attaching the device to a patient.

17. A device according to claim 16, wherein the adhesive attachment means comprises an adhesive tape or dressing attached to the head.

18. A device according to claim 16, wherein the attachment means comprises an adhesive dressing which comprises at least one wing for adhering to a patients skin.

19. A device according to claim 16, wherein the attachment means comprises an adhesive dressing which is arranged to stretch in a radial direction relative to the stem.

20. A device according to claim 19, wherein the dressing comprises a plurality of elongate slits.

21. A method of maintaining a stoma in a patient's body employing a stoma device according to claim 1, the method comprising inserting the stem until the head abuts the skin adjacent the stoma and then adhering the device to the skin.

22. A medical dressing having elongate slits formed therein such that, in use, the dressing can be stretched in a direction substantially transverse to the longitudinal extent of the slits.

23. A dressing according to claim 26, wherein the dressing comprises a plurality of slits lying on a first line and a plurality of slits lying on one or more further lines which extend generally parallel to the first line.
